(19) United States
(12) Patent Application Publication
(10) Pub. No.: US 2008/0199411 A1
(43) Pub. Date: Aug. 21, 2008

(54) Cosmetic Composition Comprising as Antiperspirant Agent a Flocculating Water-Soluble Polymer; Process For Treating Perspiration

(76) Inventors: Cyril Lemoine, Saint Cyr L' Ecole (FR); Nathalie Beau, Eragny-sur-Oise (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/660,371

(22) PCT Filed: Jun. 21, 2005
(86) PCT No.: PCT/EP2005/007370

§ 371 Date: Nov. 7, 2007

Related U.S. Application Data
(60) Provisional application No. 60/613,517, filed on Sep. 28, 2004.

(30) Foreign Application Priority Data
Aug. 18, 2004 (FR) 0451867

(51) Int. Cl. A61K 8/41; A61K 8/02; A61Q 15/00

(52) U.S. Cl. 424/47; 424/65

(57) ABSTRACT

The invention relates to a cosmetic composition comprising at least one aqueous phase and at least one antiperspirant active agent, characterized in that the said antiperspirant active agent consists of at least one flocculating water-soluble polymer comprising amine groups on the main chain and in that it does not contain any aluminium and/or zirconium antiperspirant salts.

The present invention also relates to a cosmetic process for treating perspiration, which consists in applying to the surface of the skin an effective amount of such a composition.

The present invention also relates to the use of a flocculating water-soluble polymer comprising amine groups on the main chain as an antiperspirant active agent in a cosmetic composition, and in particular in a cosmetic composition not containing any aluminium and/or zirconium antiperspirant salts.

The invention relates to a cosmetic composition comprising at least one aqueous phase and an antiperspirant active agent, characterized in that the said antiperspirant active agent consists of at least one flocculating water-soluble polymer comprising amine groups on the main chain and in that it does not contain any aluminium and/or zirconium antiperspirant salts.

The present invention also relates to a cosmetic process for treating perspiration, which consists in applying to the surface of the skin an effective amount of such a composition.

It is well known in cosmetics to use in topical application antiperspirant products containing substances that have the effect of limiting or even suppressing the flow of sweat. These products are generally available in the form of roll-ons, sticks, aerosols or sprays.

Antiperspirant substances generally consist of aluminium salts or aluminium/zirconium salts. Their antiperspirant efficacy is limited when they are used alone. The use of these active agents at high concentrations to obtain good efficacy results in formulation difficulties. Furthermore, these substances have an irritant potential to the skin.

There is thus a need to find novel antiperspirant active agents that can replace aluminium salts and aluminium/zirconium salts, and that are effective and easy to formulate.

Water-insoluble polymers forming an occlusive film on the skin have already been proposed in patent application WO 95/24105 as antiperspirant active agents. It is not necessary to use standard aluminium salts. The occlusive polymers proposed are of the octacrylamide/acrylates copolymer type or of the vinyl acetate/butyl maleate/isobornyl acrylates copolymer type, alone or in combination with a PVP/linear α-olefin polymer, for instance PVP/eicosene.

Water-insoluble film-forming polymers whose main chain is hydrocarbon-based and which comprise pendent hydrophobic quaternary ammonium groups have also been proposed in patent application WO 95/27473 as antiperspirant active agents.

Patent application WO 01/54658 discloses anhydrous compositions containing a cyanoacrylate monomer that reacts with sweat to form in situ by polymerization a film on the skin that blocks the sweat ducts.

However, these occlusive film-forming polymers do not make it possible to obtain fully satisfactory antiperspirant efficacy and still elicit formulation problems.

Moisture-absorbing polymers have been proposed as substitutes for standard astringent salts in antiperspirant anhydrous aerosol compositions in patents U.S. Pat. No. 4,743,440 and U.S. Pat. No. 4,822,596. These moisture-absorbing polymers may especially be water-soluble and chosen especially from ionenes, natural gums (xanthan, agar, carrageenans, guar or gelatin), celluloses (hydroxypropylmethylcellulose, carboxymethylcellulose), polyoxyethylenes, polyvinylpyrrolidones, polycarboxyvinyl polymers or vinyl ether/maleic anhydride copolymers. The ionene-based anhydrous aerosol compositions described in the said document are not satisfactory as regards the antiperspirant efficacy.

In patent application WO 03/030 853, the recommended moisture-absorbing polymers are chosen from grafted starch homopolymers and 2-propenamide-co-propenoic acid sodium salt copolymers.

However, these moisture-absorbing polymers do not make it possible to obtain fully satisfactory antiperspirant efficacy and still elicit formulation problems.

Water-soluble quaternary polymers have been proposed in antiperspirant compositions in the presence of standard aluminium salts to improve their efficacy. This is the case for dimethyldiallylammonium chloride in patent application EP 222 580, which acts as an agent for retaining the antiperspirant salt. This is the case for the water-soluble polymers comprising a Brönsted acid in patent application WO 02/49590, in particular those derived from maleic acid and/or maleic anhydride, which act as co-gelling agent with the antiperspirant salts. This is the case for polyethyleneimines (PEI) in the article Cosmetics & Toiletries Vol. 108 August 1993 pages 73-77, which act as completing agents for the aluminium salts.

Dimethyldiallylammonium chloride/acrylic acid copolymers have been proposed in patent application EP 478 327 as thickeners in aqueous liquid antiperspirant products containing aluminium salts.

In patent U.S. Pat. No. 4,690,817, film-forming polymers of vinyl alcohols containing pendent quaternary amine groups have been proposed in antiperspirant compositions in the presence of standard astringent salts as skin conditioners, forming a moisturizing barrier thereon.

In patent application WO 82/01993, poly-ethyleneimines have been used as odour absorbers in particular of fatty acids, aldehydes or ketones and more particularly in alcohol-based deodorant products in spray or roll-on form. The said document does not describe or suggest any activity of these polymers on the flow of sweat. The said document does not describe or deal with the moisture problems of perspiration.

The Applicant has discovered, surprisingly, that flocculating water-soluble cationic polymers comprising amine groups on the main chain constitute by themselves excellent antiperspirant agents and can be easily formulated in numerous products for treating perspiration, without it being necessary to use standard astringent salts.

One subject of the invention is a cosmetic composition comprising at least one aqueous phase and an antiperspirant active agent, characterized in that the said antiperspirant active agent consists of at least one flocculating water-soluble polymer comprising amine groups on the main chain and in that it does not contain any aluminium and/or zirconium antiperspirant salts.

A subject of the present invention is also a cosmetic process for treating perspiration, which consists in applying to the surface of the skin an effective amount of such a composition.

A subject of the present invention is also the use of a flocculating water-soluble polymer comprising amine groups on the main chain as antiperspirant active agent in a cosmetic composition, and in particular in a cosmetic composition comprising an aqueous phase.

A subject of the present invention is also the use of a flocculating water-soluble polymer comprising amine groups on the main chain as an antiperspirant active agent in a composition not containing any aluminium and/or zirconium antiperspirant salts.

The term “antiperspirant agent” means any substance that has the effect of reducing or limiting the flow of sweat.

The term “water-soluble polymer” means polymers which, when introduced into an aqueous phase at 25° C., at a mass concentration equal to 1%, make it possible to obtain a macroscopically homogeneous and transparent solution, i.e. a solution that has a minimum light transmittance value, at a wavelength equal to 500 nm, through a sample 1 cm thick, of at least 80% and preferably of at least 90%.

The term “flocculant” means any substance capable of destabilizing a colloidal suspension indistinctly via a flocculation or coagulation mechanism. The term “destabilization of colloids”means the formation of aggregates that make the suspension unstable. Since the terms flocculation and coagulation are generally interchangeable and equivalent, we will use the term “flocculation”in the invention to refer to either mechanism. The definitions of these mechanisms are given in Volume 10 of “Encyclopedia of Chemical Technology”; Kirk-Othmer; 3rd edition.

From an experimental point of view, according to the invention, a flocculant will be considered as effective in antiperspirant terms if it reduces by at least 10% and better still 20% the transmittance measured at a wavelength of 700 nm of a solution of natural sweat filtered through a 200 micron filter.

The term “composition not containing any aluminium and/or zirconium antiperspirant salts” means any composition containing not more than 1% by weight of aluminium and/or zirconium antiperspirant salt.

The term “composition comprising at least one aqueous phase” means a cosmetic composition comprising at least 1% water and preferably at least 10% water.

Flocculating water-soluble polymers comprise on the main chain amine functions partially or totally ionized in quaternary amine form.

According to the invention, the term “polymers with amines partially ionized in quaternary amine form” means any polymer for which at least one amine of the main chain is in quaternary amine form between pH 4 and pH 8.

The flocculating water-soluble polymers are preferably chosen from:
(i) polyhydroxyalkyleneamines;
(ii) polyalkyleneamines;
(iii) polyamidopolyamines;
(iv) polyamines as defined in the modified paragraphs (i), (ii) or (iii).

### A—Polyhydroxyalkyleneamines

Such polymers may be derived from the reaction of an epihalohydrin (preferably epichloro-hydrin) with a primary or secondary amine (preferably dimethylamine, diethylamine, methylethylamine, monoethylamine or monomethylamine). This type of polymer may be in linear or branched form. Furthermore, the said polymer is preferably in quaternary amine form.

Use will preferably be made of those corresponding to the general formula (I) below

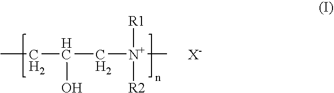

in which R1 and R2 are identical or different groups of C₁-C₄ alkyl, amine or hydrogen type; X⁻ is a mineral anion, for instance a halide (bromide, chloride or iodide) or an organic anion; n is an integer ranging from 3 to 20 000 and preferably less than 1000.

Their molecular weight (g/mol) preferably ranges from 1000 to 2 000 000 and preferably less than 100 000.

One particular family of polyhydroxyalkylene-amines in accordance with the invention are those chosen from
a) those of formula (IA) below:

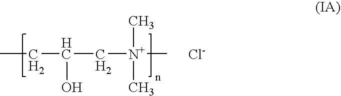

with n being an integer ranging from 3 to 20 000 and preferably less than 1000, for instance the commercial products Floquat FL 12 and FL 14 sold by the company SNF;
b) or those of formula (IB) below:

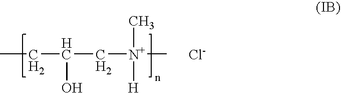

with n being an integer ranging from 3 to 20 000 and preferably less than 1000, for instance the commercial product Busan 1055 from Buckman Laboratories Inc.

### B—Polyalkyleneimines

Such polymers may be derived from the polymerization reaction using an alkyleneimine (preferably ethyleneimine). Such polymers are commonly known as polyalkyleneimines.

Such polymers may also be derived from the condensation reaction of an alkyldiamine with a dihalo-alkyl derivative.

The polyalkyleneimines used according to the invention are polymers containing from 6 to 20 000 repeating units. Preferably, polyalkyleneimines comprising at least 5% of tertiary amines, advantageously at least 10% and even more preferably at least 20% of tertiary amine functions, are chosen. These polymers may be homopolymers or copolymers, which are linear, branched or of dendrimeric structure.

The polyalkyleneimines used in accordance with the invention generally have a weight-average molecular weight ranging from 300 to 100 000, preferably from 350 to 50 000, more particularly from 400 to 10 000 and preferably from 500 to 2000. The molecular weights may be determined by quasi-elastic light scattering.

Polyalkyleneimines are especially described in the book “Polymer Science Dictionary” 2nd edition, Mark Alger, Chapman & Hall, 1997, and also in the document “The polymerization of olefin imines” by Jones, in P.H. Plesch ed., The chemistry of cationic polymerization, New York, MacMillan (1963) pages 521-534.

The general form of the alkyleneimine homopolymers corresponds to the structure:

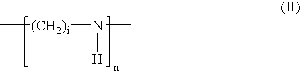

in which i is an integer ranging from 2 to 20 approximately and preferably less than 6, and n is an integer ranging from 10 to 10⁶ and preferably from 10 to 10⁴.

One particular and preferred form corresponds to the ethyleneimine homopolymers having the structure:

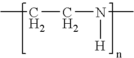

n is an integer ranging from 10 to 10⁶ and preferably from 10 to 10⁴.

The alkyleneimine homopolymers are linear or branched and defined with ratios of primary, secondary and tertiary amine. A partial description of the structure gives:

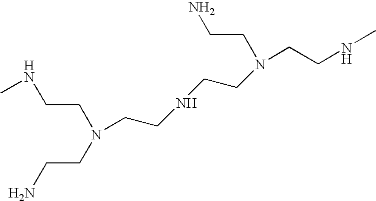

These polyalkyleneamines derived from polycondensation reactions may be chosen from those of formula (III) below:

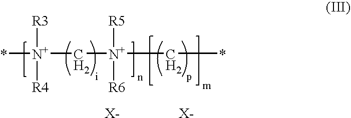

in which:
R3, R4, R5 and R6 are identical or different and denote a C1-C4 alkyl or hydroxyalkyl radical;
i and p are integers ranging from 2 to 20 approximately and preferably less than 6;
m and n are integers ranging from 1 to 10⁴ and preferably from 1 to 100;
X⁻ is an anion as described above.

Among these compounds, use will be made more particularly of the one for which R1=R2=R3=R4=CH₃; i=3; p=6, m=n and X═Cl, known as hexadimethrine chloride according to the INCI nomenclature and manufactured by Chimex under the trade name Mexomere PO.

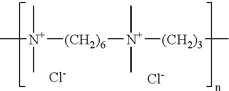

Examples that may be mentioned include the following polyethyleneimines (INCI name: PEI): PEI-7, PEI-10, PEI-15, PEI-30, PEI-35, PEI-45, PEI-250, PEI-275, PEI-700, PEI-1000, PEI-1400, PEI-1500, PEI-1750, PEI-2500, PEI-14000.

These polyethyleneimines are sold under the following trade names by the company BASF:
Lupasol FG (PEI-10): the primary/secondary/tertiary amine ratios are: 42/35/23, the molar mass being 800 g/mol,
Lupasol G-35 (PEI-35): the primary/secondary/tertiary amine ratios are: 38/36/26, the molar mass being 2000 g/mol,
Lupasol Water Free (PEI-250): the primary/secondary/-tertiary amine ratios are: 34/40/26, the molar mass being 25 000 g/mol,
Lupasol P and Lupasol PS (PEI-1500): the primary/-secondary/tertiary amine ratios are: 35/38/27, the molar mass being 750 000 g/mol.

These polyethyleneimines are sold under the trade name Epomin by the company Aceto Corporation:
Epomin SP-006 (PEI-15); Epomin SP-012 (PEI-30); Epomin SP 015 (PEI-45), Epomin P1000 (PEI-1750); Epomin P1010.

### C—Polyamidopolyamines (PAPA)

The polyaminoamides according to the invention may be chosen from those prepared by the polycondensation of an acidic compound with a polyamine. Such polymers are described, for example, in patents FR 2 252 840 and FR 2 442 632.

These polyamidopolyamines preferably correspond to formula (IV) below:

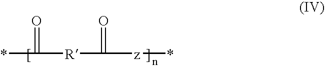

in which R′ denotes one of the following divalent groups:

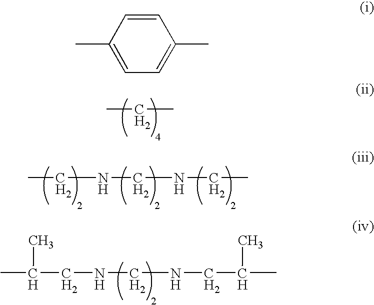

and Z represents
a) in proportions of 60 to 100 mol %, the radical:

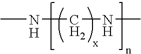

in which: x=2 and n=2 or 3, or alternatively x=3 and n=2,
b) in proportions of 0 to 40 mol %, a radical taken from the group consisting of

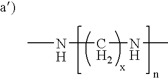

in which x=2 and n=1

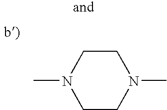

c) in proportions of 0 to 20 mol %, the radical

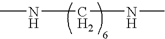

### D—Modified Polyamines

The polyamines as described previously in paragraphs A, B or C according to the invention may be modified by crosslinking or addition reaction of the non-quaternized amines present on the main chain of the said polymers with compounds comprising functional groups capable of reacting with the said amines, for instance monocarboxylic or polycarboxylic acids, dianhydrides, ketones or aldehydes, isocyanates or isothiocyanates, alkylene oxides, alkylene oxide homopolymers or copolymers, epoxides and more particularly diepoxides, esters, polyvinyl alcohols and especially divinyl sulfone, epihalohydrins and bis-halohydrins.

These polymers are preferably obtained by reacting a polyalkylene polyamine comprising two primary amine groups and at least one secondary amine group with a dicarboxylic acid chosen from diglycolic acid and saturated aliphatic dicarboxylic acids containing from 3 to 8 carbon atoms, the resulting polyamidopolyamine (PAPA) being reacted with an epichlorohydrin. Such polymers are especially described in patents U.S. Pat. No. 3,227,615 and U.S. Pat. No. 2,961,347.

Polymers of this type are sold in particular under the name “Hercosett® 57” by the company Hercules Inc. or alternatively under the name “PD 170” or “Delsette® 101” by the company Hercules in the case of the adipic acid/epoxypropyl/diethylenetriamine copolymer (INCI name: adipic acid/epoxypropyl/diethyl-enetriamine copolymer) of structure:

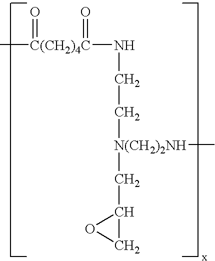

By way of example of a polyamidopolyamine modified with an epichlorohydrin, mention may be made of the products obtained by polymerization of the following monomers
diethylenetriamine (DETA)
tetraethylenepentamine (TEPA)
adipic acid

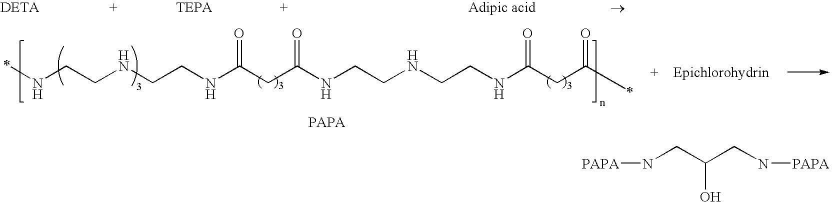

The modified polyamidopolyamines according to the invention may be chosen from those resulting from the condensation of polyalkylene polyamines with polycarboxylic acids, followed by an alkylation with difunctional agents such as those described in patent FR 1 583 363.

Mention may be made in particular of the adipic acid/dimethylaminohydroxypropyl/diethylene-triamine polymers (INCI name: Adipic acid/dimethyl-aminohydroxypropyl diethylenetriamine copolymer) sold under the trade name Cartaretine® F, F4 and F8 by the company Clariant, and of structure:

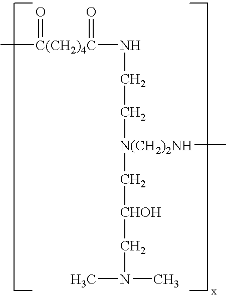

Examples that may also be mentioned include the polyethyleneimines modified with ethoxylated groups, sold under the name Lupasol SC61 by the company BASF, of structure

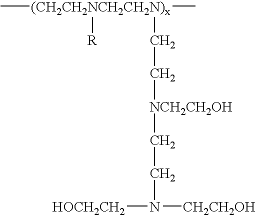

in which R denotes hydrogen or a continuation of the polymer chain. The average molecular weight is 110 000 g/mol.

The flocculating water-soluble polymers used as antiperspirant active agents are preferably present in the compositions according to the invention in amounts ranging from 0.1% to 50% and more preferably from 1% to 20% by weight relative to the total weight of the composition.

According to one particular form of the invention, the antiperspirant compositions comprise at least 5% of flocculating water-soluble polymer as described above.

The compositions according to the invention intended for cosmetic use may be in the form of lotions, creams or fluid gels distributed as an aerosol spray, in a pump-dispenser bottle or as a roll-on, in the form of thick creams distributed in tubes or a grille; in the form of wands (sticks), and may contain in this regard the ingredients generally used in products of this type and well known to those skilled in the art.

The compositions according to the present invention intended for cosmetic use may comprise at least one aqueous phase. They are especially formulated as aqueous lotions or as water-in-oil, oil-in-water emulsions, or as multiple emulsions (oil-in-water-in-oil or water-in-oil-in-water triple emulsions (such emulsions are known and described, for example, by C. Fox in “Cosmetics and Toiletries”, November 1986, Vol. 101, pages 101-112)).

The aqueous phase of the said compositions contains water and generally other water-soluble or water-miscible solvents. The water-soluble or water-miscible solvents include short-chain monoalcohols, for example of C₁-C₄, for instance ethanol or isopropanol; diols or polyols, for instance ethylene glycol, 1,2-propylene glycol, 1,3-butylene glycol, hexylene glycol, diethylene glycol, dipropylene glycol, 2-ethoxyethanol, diethylene glycol monomethyl ether, triethylene glycol monomethyl ether and sorbitol. Propylene glycol and glycerol will more particularly be used.

According to one particular form of the invention, the antiperspirant compositions comprise at least 5% of flocculating polymer as described above.

The compositions according to the invention preferably comprise at least one water-immiscible organic liquid phase.

This phase generally comprises one or more hydrophobic compounds that render the said phase water-immiscible. The said phase is liquid (in the absence of a structuring agent) at room temperature (20-25° C.). Preferentially the water-immiscible organic liquid phase in accordance with the invention generally consists of an oil or a mixture of oils and comprises at least 80% of compounds with a vapour pressure not exceeding 4 kPa (30 mmHg) at 25° C.

The water-immiscible organic liquid phase preferably contains one or more volatile or non-volatile, silicone-based or hydrocarbon-based emollient oils. These emollient oils are especially described in patents U.S. Pat. No. 4,822,596 and U.S. Pat. No. 4,904,463.

Volatile silicones are defined, in a known manner, as being compounds that are volatile at room temperature. Mention may be made among these compounds of cyclic and linear volatile silicones of the dimethylsiloxane type whose chains comprise from 3 to 9 silicone-based residues. Cyclomethicones D₄, D₅ or D₆ are preferably chosen.

Non-volatile silicones are defined, in a known manner, as being compounds with a low vapour pressure at room temperature. The following are included among these compounds: polyalkylsiloxanes, in particular linear polyalkylsiloxanes, for instance the linear polydimethylsiloxanes, or dimethicones, sold by the company Dow Corning under the name “Dow Corning 245 Fluid”; polyalkylarylsiloxanes, for instance the polymethylphenylsiloxanes sold by the company Dow Corning under the name “Dow Corning 556 Fluid”; copolymers of polyether and siloxane, for instance dimethicone copolyols.

Among the non-volatile emollient oils that may be used in the present invention, examples that may be mentioned include: hydrocarbon-based derivatives, mineral oils, fatty alcohols, esters of C₃-C₁₈ alcohols with C₃-C₁₈ acids, esters of benzoic acid with C₁₂-C₁₈ alcohols and mixtures thereof, C₂-C₆ polyols preferably chosen from glycerol, propylene glycol or sorbitol, polyalkylene glycol polymers.

The emollient oils are preferably present in amounts ranging from 1% to 50% by weight and more preferably from 5% to 40% by weight relative to the total weight of the composition.

The cosmetic composition according to the invention may contain one or more additional deodorant active agents, for instance bacteriostatic agents or bactericidal agents such as 2,4,4′-trichloro-2′-hydroxydiphenyl ether (Triclosan), 2,4-dichloro-2′-hydroxydiphenyl ether, 3′,4′,5′-trichlorosalicyl-anilide, 1-(3′,4′-dichlorophenyl)-3-(4′-chlorophenyl)-urea (Triclocarban) or 3,7,11-trimethyldodeca-2,5,10-trienol (Farnesol); quaternary ammonium salts, for instance cetyltrimethylammonium salts or cetylpyri-dinium salts; chlorhexidine and salts; diglyceryl monocaprate, diglyceryl monolaurate or glyceryl monolaurate; polyhexamethylene biguanide salts.

In order to improve the antiperspirant efficacy of the composition, one or more water-soluble anionic polymers comprising a Brönsted acid, in particular those derived from maleic acid and/or from maleic anhydride, which are described in patent application WO 02/49590, may also be used.

In order to improve the homogeneity of the product, it is possible also to use one or more suspenders preferably chosen from hydrophobic-modified montmorillonite clays, for instance hydrophobic-modified bentonites or hectorites. Examples that may be mentioned include the product stearalkonium bentonite (CTFA name) (product of reaction of bentonite and the quaternary ammonium stearalkonium chloride), such as the commercial product sold under the name Tixogel MP 250 by the company Süd Chemie Rheologicals, United Catalysts Inc. or the product disteardimonium hectorite (CTFA name) (product of reaction of hectorite and of distearyldimonium chloride) sold under the name Bentone 38 or Bentone Gel by the company Elementis Specialities.

The suspenders are preferably present in amounts ranging from 0.1% to 5% by weight and more preferably from 0.2% to 2% by weight relative to the total weight of the composition.

The compositions according to the invention may also contain at least one organic powder.

Among the fillers that may be used according to the invention, mention may be made of organic powders. In the present patent application, the term “organic powder” means any solid that is insoluble in the medium at room temperature (25° C.).

As organic powders that may be used in the composition of the invention, examples that may be mentioned include polyamide particles and especially those sold under the name Orgasol by the company Atochem; polyethylene powders; microspheres based on acrylic copolymers, such as those made of ethylene glycol dimethacrylate/lauryl methacrylate copolymer, sold by the company Dow Corning under the name Polytrap; polymethyl methacrylate microspheres, sold under the name Microsphere M-100 by the company Matsumoto or under the name Covabead LH85 by the company Wackherr; ethylene-acrylate copolymer powders, for instance those sold under the name Flobeads by the company Sumitomo Seika Chemicals; expanded powders such as hollow microspheres and especially microspheres formed from a terpolymer of vinylidene chloride, of acrylonitrile and of methacrylate and sold under the name Expancel by the company Kemanord Plast under the references 551 DE 12 (particle size of about 12 μm and density of 40 kg/m³), 551 DE 20 (particle size of about 30 μm and a density of 65 kg/m³) and 551 DE 50 (particle size of about 40 μm), or the microspheres sold under the name Micropearl F 80 ED by the company Matsumoto; powders of natural organic materials such as starch powders, especially of corn starch, wheat starch or rice starch, which may or may not be crosslinked, such as the starch powder crosslinked with octenylsuccinate anhydride, sold under the name Dry-Flo by the company National Starch; silicone resin microbeads such as those sold under the name Tospearl by the company Toshiba Silicone, especially Tospearl 240; amino acid powders such as the lauroyllysine powder sold under the name Amihope LL-11 by the company Ajinomoto; particles of wax microdispersion, which preferably have mean sizes of less than 1 μm and especially ranging from 0.02 μm to 1 μm, and which consist essentially of a wax or a mixture of waxes, such as the products sold under the name Aquacer by the company Byk Cera, and especially: Aquacer 520 (mixture of synthetic and natural waxes), Aquacer 514 or 513 (polyethylene wax), Aquacer 511 (polymer wax), or such as the products sold under the name Jonwax 120 by the company Johnson Polymer (mixture of polyethylene wax and paraffin wax) and under the name Ceraflour 961 by the company Byk Cera (micronized modified polyethylene wax); and mixtures thereof. The organic powder(s) may be present, for example, in an amount

The cosmetic composition according to the invention may also comprise cosmetic adjuvants chosen from waxes, softeners, antioxidants, opacifiers, stabilizers, moisturizers, vitamins, fragrances, bactericides, preserving agents, polymers, fragrances, thickeners, propellants or any other ingredient usually used in cosmetics for this type of application.

Needless to say, a person skilled in the art will take care to select this or these optional additional compound(s) such that the advantageous properties intrinsically associated with the cosmetic composition in accordance with the invention are not, or are not substantially, adversely affected by the envisaged addition(s).

The waxes may be chosen from animal, fossil, plant, mineral and synthetic waxes. Mention may be made especially of beeswaxes, carnauba wax, candelilla wax, sugar cane wax, Japan wax, ozokerites, montan wax, microcrystalline waxes, paraffins and silicone waxes and resins.

The thickeners, which are preferably nonionic, may be chosen from modified or unmodified guar gums and celluloses, such as hydroxypropyl guar gum or cetylhydroxyethylcellulose, silicas, for instance Bentone Gel MIO sold by the company NL Industries, or Veegum Ultra sold by the company Polyplastic.

The amounts of these various constituents that may be present in the cosmetic composition according to the invention are those conventionally used in deodorant compositions.

The compositions according to the invention may also contain one or more other agents for structuring or gelling the water-immiscible organic liquid phase of the composition, such as linear solid fatty alcohols and/or waxes; fatty acids or salts thereof (stearic acid, sodium stearate or 12-hydroxy-stearic acid; dibenzylidene alditols (DBS); lanosterol, N-acylamino acid derivatives; di- or tricarboxylic acid derivatives, for instance alkyl-N,N′-dialkylsuccin-amides (i.e.: dodecyl-N,N′-dibutylsuccinamide); elastomeric polyorganosiloxanes such as those described in patent application WO 97/44010.

The composition according to the invention may also be pressurized and may be packaged in an aerosol device.

A subject of the present invention is an aerosol device consisting of:
(A) a container comprising a deodorant composition as defined above,
(B) at least one propellant and a means for distributing the said aerosol composition.

The propellants generally used in products of this type, which are well known to those skilled in the art, are, for example, dimethyl ether (DME); volatile hydrocarbons such as n-butane, propane or isobutane, and mixtures thereof, optionally with at least one chlorohydrocarbon and/or fluorohydrocarbon; among the latter, mention may be made of the compounds sold by the company Dupont de Nemours under the names Freon® and Dymel®, and in particular monofluorotrichloro-methane, difluorodichloromethane, tetrafluorodichloro-ethane and 1,1-difluoroethane sold especially under the trade name Dymel 152 A by the company Dupont. Carbon dioxide, nitrous oxide, nitrogen or compressed air may also be used as propellant.

The composition containing the deodorant active agent(s) and the propellant(s) may be in the same compartment or in different compartments in the aerosol container. According to the invention, the concentration of propellant generally ranges from 5% to 95% by pressurized weight and more preferably from 50% to 85% by weight relative to the total weight of the pressurized composition.

The distribution means, which forms a part of the aerosol device, generally consists of a distribution valve controlled by a distribution head, itself comprising a nozzle via which the aerosol composition is vaporized. The container containing the pressurized composition may be opaque or transparent. It may be made of glass, of polymeric material or of metal, optionally coated with a layer of protective varnish.

The examples that follow serve to illustrate the present invention.

### EXAMPLE 1

### Antiperspirant Roll-On

| Phase | Ingredients | Amounts (g) |
|---|---|---|
| A | Ethoxylated polyethyleneimine: | 20 |
| | Lupasol SC61 (aqueous 35% solution) | |
| | Zinc gluconate | 2 |
| B | Steareth-21 | 2 |
| | Steareth-2 | 2 |
| | Steareth-5 stearate | 1 |
| | PPG-15 stearyl ether | 1.5 |
| | Silicone D5 | 3.5 |
| C | Water | 68 |
| | | 100 |

### Procedure:

Phases (B) and (C) are separately heated to 70° C. (B) and (C) are mixed together with a Turrax blender for 5 minutes and then cooled to 55° C. with paddle stirring. Phase A is added gently with stirring. The mixture is homogenized for 1 to 3 minutes. It is cooled to 35° C. with stirring.

### EXAMPLE 2

### Antiperspirant Vaporizer (PIT Emulsion)

| Ingredients | Amounts (g) |
|---|---|
| PEI 10-Lupasol FG | 15 |
| Cetearyl isononanoate (and) cetearyl | 15 |
| alcohol (and) Ceteareth-20 (and) glycerol | |
| (and) glyceryl stearate (and) Ceteareth- | |
| 12 (and) cetyl palmitate (Emulgade CM) | |
| Water | 70 |
| | 100 |

### Procedure:

The polyalkyleneimine is dissolved in water and the emulsifying agent is added to the mixture with moderate stirring.

1. Cosmetic composition comprising at least one aqueous phase and at least one antiperspirant active agent, characterized in that the said antiperspirant active agent consists of at least one flocculating water-soluble polymer comprising amine groups on the main chain and in that it does not contain any aluminium and/or zirconium antiperspirant salts.

2. Composition according to claim 1, in which the flocculating water-soluble polymers are polyamines chosen from:
(i) polyhydroxyalkyleneamines;
(ii) polyalkyleneamines;
(iii) polyamidopolyamines;
(iv) polyamines as defined in the modified paragraphs (i), (ii) or (iii).

3. Composition according to claim 2, in which the polyhydroxyalkyleneamines are chosen from those corresponding to the general formula (I): 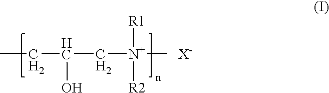
in which R1 and R2 are identical or different groups of C₁-C₄ alkyl, amine or hydrogen type; X⁻ is a mineral or organic anion; n is an integer ranging from 3 to 20 000 and preferably less than 1000.

4. Composition according to claim 3, in which the polyhydroxyalkyleneamines have a molecular weight ranging from 1000 to 2 000 000 g/mol and preferably less than 100 000.

5. Composition according to claim 3 or 4, in which the polyhydroxyalkyleneamines are chosen from:
a) those of formula (IA) below: 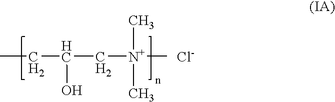
b) those of formula (IB) below: 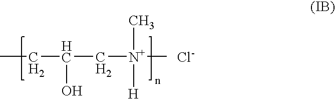
with n being an integer ranging from 3 to 20 000 and preferably less than 1000.

5. Composition according to claim 2, in which the polyalkyleneamines are polyalkyleneimines.

6. Composition according to claim 5, in which the polyalkyleneamines contain from 6 to 20 000 repeating units.

7. Composition according to claim 5 or 6, in which the polyalkyleneimines comprise at least 5% of tertiary amines, advantageously at least 10% and even more preferably at least 20% of tertiary amine functions.

8. Composition according to any one of claims 5 to 7, in which the polyalkyleneimines have a weight-average molecular weight ranging from 300 to 100 000, preferably from 350 to 50 000, more particularly from 400 to 10 000 and preferably from 500 to 2000.

9. Composition according to any one of claims 5 to 8, in which the polyalkyleneimines are chosen from those of structure: 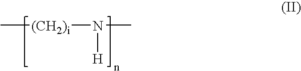
in which i is an integer ranging from 2 to 20 approximately and preferably less than 6, and n is an integer ranging from 10 to 106 and preferably from 10 to 10⁴.

10. Composition according to claim 9, in which the polyalkyleneimines are polyethyleneimines of structure: 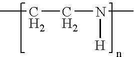
in which n is an integer ranging from 10 to 10⁶ and preferably from 10 to 10⁴.

11. Composition according to claim 5, in which the polyalkyleneamines are chosen from those of formula (III) below: 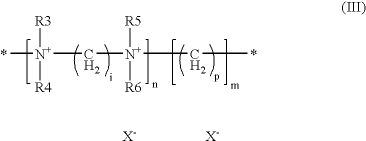
in which:
R3, R4, R5 and R6 are identical or different and denote a C1-C4 alkyl or hydroxyalkyl radical;
i and p are integers ranging from 2 to 20 approximately and preferably less than 6;
m and n are integers ranging from 1 to 10⁴ and preferably from 1 to 100;
X⁻ is a mineral or organic anion.

12. Composition according to claim 11, in which the polyalkyleneamine of formula (III) is the polymer for which R1, R2, R3 and R4 simultaneously denote methyl; i=3; p=6 and X═Cl (hexadimethrine chloride) of structure:

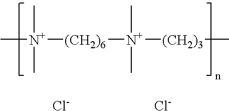

13. Composition according to claim 2, in which the polyamidopolyamines correspond to formula (IV) below 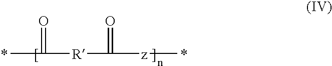
in which R′ denotes one of the following divalent groups: 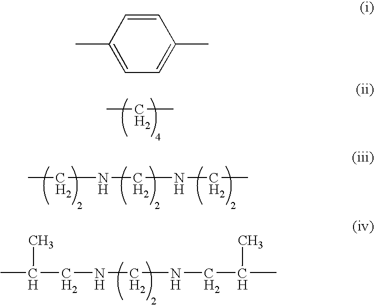
and Z represents
a) in proportions of 60 to 100 mol %, the radical: 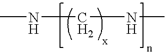
in which: x=2 and n=2 or 3, or alternatively x=3 and n=2,
b) in proportions of 0 to 40 mol %, a radical taken from the group consisting of 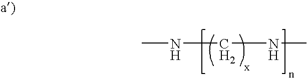
in which x=2 and n=1 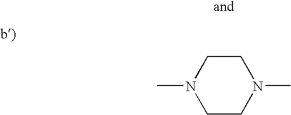
c) in proportions of 0 to 20 mol %, the radical

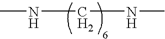

14. Composition according to claim 2, in which the polyamines as defined in the modified paragraphs (i), (ii) or (iii) are obtained by crosslinking or addition reaction of the non-quaternized amines present on the main chain of the said polyamines with compounds comprising functional groups capable of reacting with the said amines, in particular monocarboxylic or polycarboxylic acids, dianhydrides, ketones or aldehydes, isocyanates or isothiocyanates, alkylene oxides, alkylene oxide homopolymers or copolymers, and epoxides.

15. Composition according to claim 14, in which the compounds comprising functional groups are chosen from diepoxides, esters, polyvinyl alcohols and especially divinyl sulfone, epihalohydrins and bis-halohydrins.

16. Composition according to claim 14 or 15, in which the modified polyamines are chosen from polyamidopolyamines modified with an epichlorohydrin.

17. Composition according to claim 16, in which the polyamidopolyamine modified with an epichlorohydrin is an adipic acid/epoxypropyl/diethyl-enetriamine copolymer (INCI name: adipic acid/epoxypropyl/diethylenetriamine copolymer) of structure:

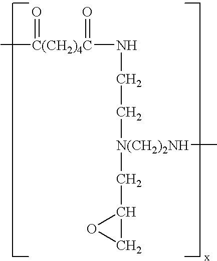

18. Composition according to claim 16, in which the polyamidopolyamine modified with an epichlorohydrin is obtained by polymerization of the following monomers
diethylenetriamine (DETA)
tetraethylenepentamine (TEPA)
adipic acid.

19. Composition according to claim 16, in which the modified polyamidopolyamines are chosen from those resulting from the condensation of polyalkylene polyamines with polycarboxylic acids followed by an alkylation with difunctional agents.

20. Composition according to claim 16, in which the modified polyamidopolyamines are chosen from adipic acid/dimethylaminohydroxypropyl/diethylene-triamine polymers (INCI name: Adipic acid/dimethyl-aminohydroxypropyl diethylenetriamine copolymer) and of structure:

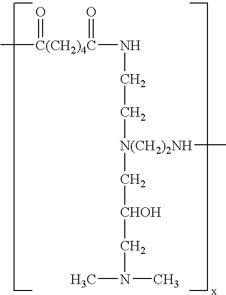

21. Composition according to claim 14 or 15, in which the modified polyamines are chosen from polyethyleneimines modified with ethoxylated groups.

22. Composition according to claim 21, in which the polyethyleneimines modified with ethoxylated groups are chosen from those of structure: 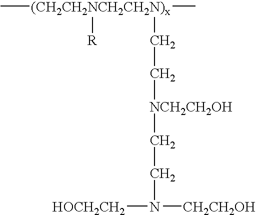
in which R denotes hydrogen or a continuation of the polymer chain.

23. Composition according to any one of claims 1 to 22, in which the flocculating water-soluble polymer is present in amounts ranging from 0.1% to 50% and more preferably from 1% to 20% by weight relative to the total weight of the composition.

24. Composition according to any one of claims 1 to 23, in which the flocculating water-soluble polymer is present in amounts of greater than 5% by weight.

25. Composition according to any one of claims 1 to 24, characterized in that it is in the form of a lotion, a cream or a fluid gel distributed as an aerosol spray, in a pump-dispenser bottle or as a roll-on; in the form of a cream or gel distributed in a tube or grille; in the form of a wand (stick).

26. Composition according to claim 25, characterized in that it is in the form of an aqueous lotion, in the form of a water-in-oil or oil-in-water emulsion; in the form of a multiple emulsion.

27. Composition according to claim 26, in which the aqueous phase contains water and one or more water-miscible or water-soluble solvents.

28. Composition according to claim 27, in which the water-miscible or water-soluble solvents are chosen from C₁-C₄ monoalcohols; diols; polyols.

29. Composition according to any one of claims 1 to 28, characterized in that it comprises at least one water-immiscible organic liquid phase.

30. Composition according to claim 29, in which the organic liquid phase comprises one or more silicone-based or hydrocarbon-based, volatile or non-volatile emollient oils.

31. Composition according to claim 30, in which the emollient oils are present in amounts ranging from 1% to 50% by weight and more preferably from 5% to 40% by weight relative to the total weight of the composition.

32. Composition according to any one of claims 1 to 31, characterized in that it also comprises one or more additional deodorant active agents.

33. Composition according to claim 32, characterized in that it also comprises one or more bacteriostatic agents or bactericides.

34. Composition according to any one of claims 1 to 33, characterized in that it also comprises one or more water-soluble anionic polymers comprising a Brönsted acid.

35. Composition according to any one of claims 1 to 34, characterized in that it also comprises one or more suspenders.

36. Composition according to any one of claims 1 to 35, characterized in that it also comprises at least one organic powder.

37. Composition according to any one of claims 1 to 36, characterized in that it also comprises at least one cosmetic additive chosen from waxes, softeners, antioxidants, opacifiers, stabilizers, moisturizers, vitamins, fragrances, bactericides, preserving agents, polymers, fragrances, thickeners and propellants.

38. Composition according to any one of claims 29 to 37, characterized in that it also comprises one or more agents for structuring or gelling the water-immiscible organic liquid phase.

39. Aerosol device consisting of:
(A) a container comprising a deodorant composition as defined according to any one of claims 1 to 38;
(B) at least one propellant and a means for distributing the said aerosol composition.

40. Cosmetic process for treating human perspiration, which consists in applying to the surface of the skin an effective amount of a composition as defined according to any one of claims 1 to 39.

41. Use of a flocculating water-soluble polymer as defined in any one of claims 1 to 21 as an antiperspirant active agent in a cosmetic composition.

42. Use according to claim 41, in which the cosmetic composition comprises at least one aqueous phase.

43. Use according to claim 41 or 42, in which the cosmetic composition does not contain any aluminium and/or zirconium antiperspirant salts.
